(19) United States
(12) Patent Application Publication
(10) Pub. No.: US 2017/0273579 A1
(43) Pub. Date: Sep. 28, 2017

(54) BLOOD PRESSURE ESTIMATION APPARATUS, SPHYGMOMANOMETER, BLOOD PRESSURE ESTIMATION SYSTEM, AND BLOOD PRESSURE ESTIMATION METHOD

(71) Applicant: KYOCERA Corporation, Kyoto (JP)

(72) Inventors: Hiroyuki MORI, Yokohama-shi (JP); Shigeru MESAKI, Sagamihara-shi (JP)

(73) Assignee: KYOCERA Corporation, Kyoto (JP)

(21) Appl. No.: 15/465,720

(22) Filed: Mar. 22, 2017

(30) Foreign Application Priority Data
Mar. 25, 2016 (JP) 2016-062493

(51) Int. Cl. A61B 5/021; A61B 5/00; A61B 5/022

(52) U.S. Cl. CPC A61B 5/02108; A61B 5/02233; A61B 5/7275; A61B 5/6824

(57) ABSTRACT

Provided are a blood pressure estimation apparatus, a sphygmomanometer, a blood pressure estimation system, and a blood pressure estimation method which allow continuous blood pressure measurement without adding stress to a subject's body. A plurality of sensors and a controller are included. The plurality of sensors each detect a pulse wave of a subject without applying, to the subject, pressure that is greater than predetermined pressure, and the controller calculates an estimated value of subject's blood pressure continuously, based on a measured value of the subject's blood pressure acquired from a sphygmomanometer and on output of the sensors.

### CROSS REFERENCE TO RELATED APPLICATIONS

This application claims priority to and the benefit of Japanese Patent Application No. 2016-62493 filed Mar. 25, 2016, the entire contents of which are incorporated herein by reference.

### TECHNICAL FIELD

The present disclosure relates to a blood pressure estimation apparatus, a sphygmomanometer, a blood pressure estimation system, and a blood pressure estimation method.

### BACKGROUND

An example of an existing measurement apparatus measures biological information from a test site, such as a wrist, of a subject.

### SUMMARY

According to one embodiment of the present disclosure, a blood pressure estimation apparatus includes a plurality of sensors that each detects a pulse wave of a subject without applying, to the subject, pressure that is greater than predetermined pressure. The blood pressure estimation apparatus also includes a controller that calculates an estimated value of subject's blood pressure continuously, based on a measured value of the subject's blood pressure acquired from a sphygmomanometer and on output of the sensors.

According to one embodiment of the present disclosure, a sphygmomanometer includes a plurality of sensors that each detects a pulse wave of a subject without applying, to the subject, pressure that is greater than predetermined pressure; and a blood pressure measurement unit. The sphygmomanometer also includes a controller that calculates an estimated value of subject's blood pressure continuously, based on a measured value of the subject's blood pressure acquired from the blood pressure measurement unit and on output of the sensors.

According to one embodiment of the present disclosure a blood pressure estimation system includes a sphygmomanometer; and a blood pressure estimation apparatus. The blood pressure estimation apparatus includes a plurality of sensors that each detects a pulse wave of a subject without applying, to the subject, pressure that is greater than predetermined pressure. The blood pressure estimation apparatus also includes a controller that calculates an estimated value of subject's blood pressure continuously, based on a measured value of the subject's blood pressure acquired from the sphygmomanometer and on output of the sensors.

According to one embodiment of the present disclosure, a blood pressure estimation method includes detecting a pulse wave of a subject by a plurality of sensors without applying, to the subject, pressure that is greater than predetermined pressure. The blood pressure estimation method also includes calculating an estimated value of subject's blood pressure continuously, based on a measured value of the subject's blood pressure acquired from a sphygmomanometer and on output of the sensors.

### BRIEF DESCRIPTION OF THE DRAWINGS

In the accompanying drawings:

FIG. 1 is a perspective view illustrating an example of configuration of a blood pressure estimation apparatus according to Embodiment 1;

FIG. 2 illustrates an example of a configuration of a sensor holder;

FIG. 3 is a functional block diagram illustrating an example of a schematic configuration of a blood pressure estimation apparatus illustrated in FIG. 1;

FIG. 4 is a schematic view illustrating an artery of a human right arm;

FIGS. 5A and 5B each illustrate an example of a pulse wave calculated based on response information acquired from a sensor unit;

FIG. 6 illustrates an example of a schematic configuration of a blood pressure estimation system including a blood pressure estimation apparatus illustrated in FIG. 1;

FIG. 7 is a flowchart illustrating an example of a procedure of estimating subject's blood pressure;

FIG. 8 is a flowchart illustrating an example of a procedure of monitoring an estimated value of subject's blood pressure;

FIG. 9 is a graph illustrating a result of experiment regarding a relation between contact pressure applied to a wrist and PWV in a short wrist's distance;

FIG. 10 illustrates average blood pressure separated by age;

FIG. 11 is a schematic view of a sphygmomanometer according to Embodiment 2;

FIG. 12 is a functional block diagram illustrating an example of a schematic configuration of a sphygmomanometer illustrated in FIG. 11; and

FIG. 13 is a flowchart illustrating an example of a procedure of monitoring blood pressure by using a sphygmomanometer illustrated in FIG. 11.

### DETAILED DESCRIPTION

In blood pressure measurement using a cuff sphygmomanometer, cuff pressure causes congestion. Accordingly, in view of stress to a subject's body, blood pressure cannot be measured continuously by using a cuff sphygmomanometer.

The present disclosure is to provide a blood pressure estimation apparatus, a sphygmomanometer, a blood pressure estimation system, and a blood pressure estimation method which allow continuous blood pressure measurement without adding stress to the subject's body.

A blood pressure estimation apparatus, a sphygmomanometer, a blood pressure estimation system, and a blood pressure estimation method according to one of embodiments of the present disclosure allow continuous blood pressure measurement without adding stress to the subject's body.

Embodiment of the present disclosure will be described below with reference to the drawings.

### Embodiment 1

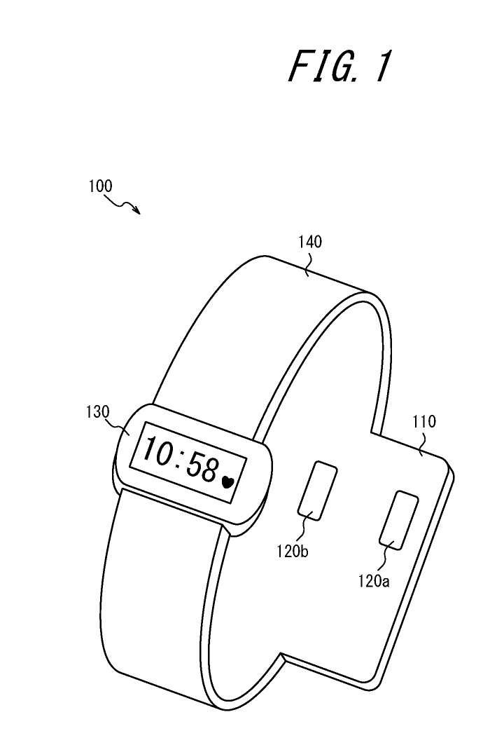

FIG. 1 is a perspective view illustrating an example of a configuration of a blood pressure estimation apparatus 100 according to Embodiment 1. The blood pressure estimation apparatus 100 includes a sensor holder 110, a display 130, and a mounted portion 140. The sensor holder 110 includes a first sensor unit 120a and a second sensor unit 120b. Each of the first sensor unit 120a and the second sensor unit 120b may also be called sensor unit 120.

The mounted portion 140 is used to mount the blood pressure estimation apparatus 100 on a subject's body (e.g., arm, wrist, or ankle). The mounted portion 140 is, for example, a band made of resin, such as rubber. The mounted portion 140 may also be in the form of a clip or the like. The mounted portion 140, which allows the blood pressure estimation apparatus 100 to be mounted on the subject's body, may include a variety of configurations.

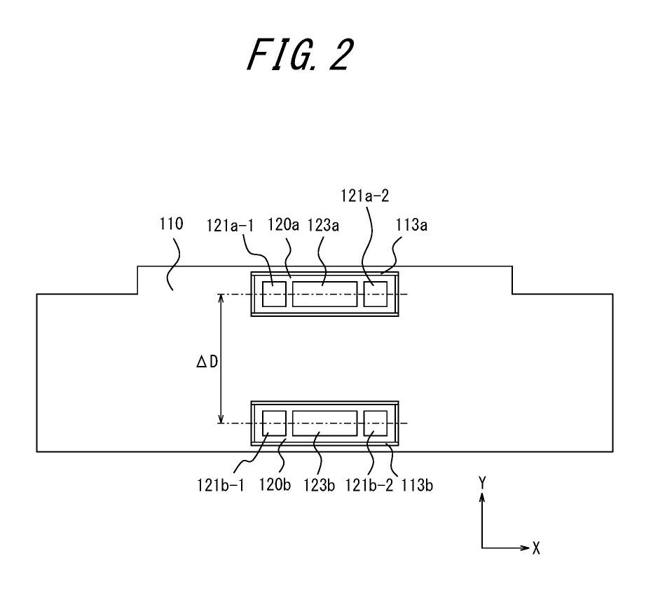

FIG. 2 illustrates an example of a configuration of the sensor holder 110. In FIG. 2, an X-Y coordinate is defined. A positive X axis direction is defined as a right direction. A positive Y axis direction is defined as an upper direction. As illustrated in FIG. 2, the sensor holder 110 includes a first opening 113a and a second opening 113b, which oppose to the subject's body. In the first opening 113a, the first sensor unit 120a is disposed. In the second opening 113b, the second sensor unit 120b is disposed.

The first sensor unit 120a and the second sensor unit 120b serve as sensors that measure biological information of the subject. The first sensor unit 120a and the second sensor unit 120b may measure the biological information of the subject while contacting a test site of the subject. In the present embodiment, the blood pressure estimation apparatus 100 includes the two sensor units 120. However, the blood pressure estimation apparatus 100 may include three or more sensor units 120. As illustrated in FIG. 2, the first sensor unit 120a and the second sensor unit 120b are disposed at a predetermined distance (ΔD) along the Y axis direction in FIG. 2. The first sensor unit 120a is disposed further in the positive Y axis direction than the second sensor unit 120b. The predetermined distance may, for example, be from 10 to 30 mm. A positional relation between the first sensor unit 120a and the second sensor unit 120b is not limited to the example illustrated in FIG. 2.

The first sensor unit 120a includes two light emitters 121a-1 and 121a-2 and a light receiver 123a. The light emitters 121a-1 and 121a-2 and the light receiver 123a are disposed along the X axis. The second sensor unit 120b includes, for example, two light emitters 121b-1 and 121b-2 and a light receiver 123b. The light emitters 121b-1 and 121b-2 and the light receiver 123b are disposed along the X axis. Each of the light emitters 121a-1, 121a-2, 121b-1, and 121b-2 may also be called light emitter 121. Each of the light receivers 123a and 123b may also be called light receiver 123. The number of the light emitters 121 included in each sensor unit 120 is not limited to two. Furthermore, the number of the light receivers 123 included in each sensor unit 120 is not limited to one. The greater the number of the light emitters 121 included in each sensor unit 120, the more precise the biological information measured by the sensor unit 120. The light emitters and the light receivers 123 may also be disposed in other ways than illustrated in the example of FIG. 2.

Each light emitter 121 includes a light emitting element, such as a Light Emitting Diode (LED) or a Laser Diode (LD). The light emitter 121 may include one, or two or more light emitting elements. Each light receiver 123 includes a light receiving element, such as a Photodiode (PD) or a Phototransistor (PT). The light receiver 123 may include one, or two or more light receiving elements.

[Functional Block]

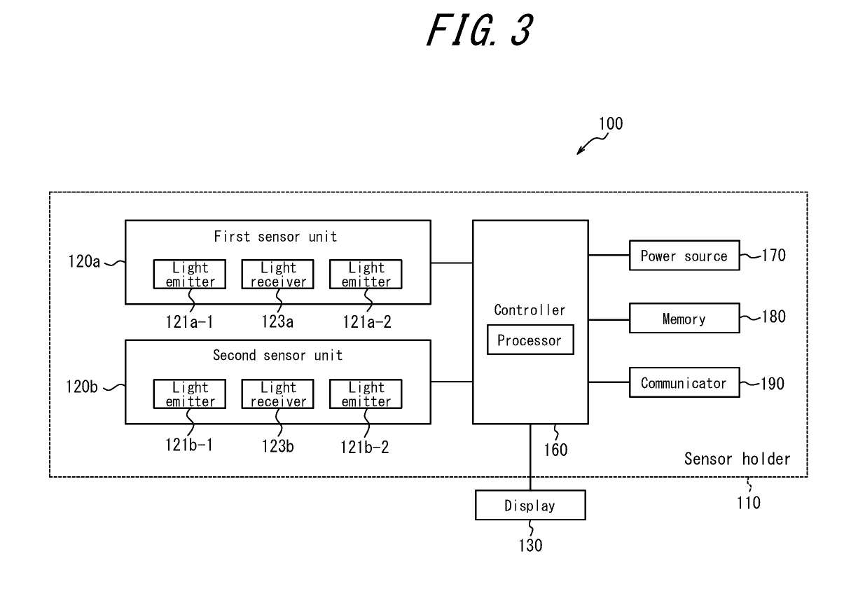

FIG. 3 is a functional block diagram illustrating an example of a schematic configuration of the blood pressure estimation apparatus 100 illustrated in FIG. 1. The blood pressure estimation apparatus 100 includes the first sensor unit 120a, the second sensor unit 120b, the display 130, a controller 160, a power source 170, a memory 180, and a communicator 190. In the present embodiment, the first sensor unit 120a, the second sensor unit 120b, the controller 160, the power source 170, the memory 180, and the communicator 190 may be configured to be included in the sensor holder 110 and the display 130.

The controller 160 is connected to function blocks of the blood pressure estimation apparatus 100. The controller 160 is a processor that controls and manages the functional blocks of the blood pressure estimation apparatus 100 and the blood pressure estimation apparatus 100 overall. The controller 160 may be a processor, such as a Central Processing Unit (CPU), that executes a program prescribing control procedures. Such a program may, for example, be stored in a storage medium, such as the memory 180.

The blood pressure estimation apparatus 100 includes at least one processor for providing control and processing capability to perform various functions as described in further detail below. In accordance with various embodiments, the at least one processor may be implemented as a single integrated circuit (IC) or as multiple communicatively coupled IC's and/or discrete circuits. It is appreciated that the at least one processor can be implemented in accordance with various known technologies. In one embodiment, the processor includes one or more circuits or units configurable to perform one or more data computing procedures or processes by executing instructions stored in an associated memory, for example. In other embodiments, the processor may be implemented as firmware (e.g. discrete logic components) configured to perform one or more data computing procedures or processes. In accordance with various embodiments, the processor may include one or more processors, controllers, microprocessors, microcontrollers, application specific integrated circuits (ASICs), digital signal processors, programmable logic devices, field programmable gate arrays, or any combination of these devices or structures, or other known devices and structures, to perform the functions described herein.

The controller 160 acquires the biological information of the subject measured by the sensor units 120. Based on the biological information of the subject, the controller 160 may calculate an estimated value of subject's blood pressure. A method of calculating the estimated value of the subject's blood pressure is described later below. When the controller 160 calculates the estimated value of the subject's blood pressure, the controller 160 constitutes an estimation unit.

The power source 170 supplies power to the blood pressure estimation apparatus 100 as a whole. The power source 170 includes, for example, a lithium-ion battery, a control circuit for charging and discharging the lithium-ion battery, and the like. The power source 170 may also be a circuit through which power is supplied from an external power source.

The memory 180 may be configured with a semiconductor memory, a magnetic memory, or the like. The memory 180 stores various information and programs for operating the blood pressure estimation apparatus 100. The memory 180 also functions as a working memory. The memory 180 may store therein the biological information acquired from the first sensor unit 120a and the second sensor unit 120b.

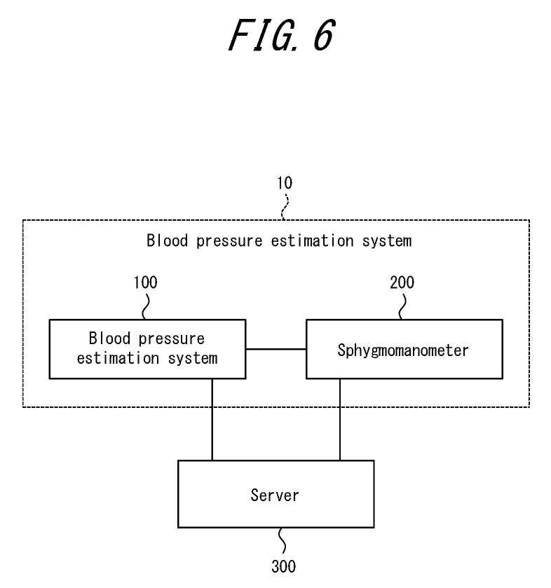

The communicator 190 transmits and receives various data to and from an external apparatus, such as a server 300 (refer to FIG. 6), through wired or wireless communication. For example, the communicator 190 communicates with an external apparatus, such as a server, that stores the biological information of the subject and transmits, to the external apparatus, the biological information measured by the blood pressure estimation apparatus 100.

The display 130 displays the estimated value or the like of the subject's blood pressure calculated by the controller 160. The display 130 may, for example, be a liquid crystal, an Electro-Luminescence (EL), an inorganic EL, or a Light Emission Diode (LED) display.

<Control of Sensor Units>

The controller 160 outputs, to each of the sensor units 120, control information for causing the corresponding light emitters 121 to emit measuring light. The control information for causing measuring light emission is, for example, a single for applying voltage to the LEDs or LDs. The controller 160 acquires, from the sensor unit 120, response information pertaining to light received by the light receiver 123. The response information pertaining to the received light is, for example, a voltage signal that the PD or the PT outputs.

When the blood pressure estimation apparatus 100 is mounted on the subject's body, the measuring light emitted from the light emitters 121 of the sensor unit 120 irradiate the test site of the subject. The measuring light is scattered from the test site. The light (scattered light) scattered from the test site enters the light receiver 123. The light receiver 123 outputs the response information pertaining to the received scattered light. The controller 160 is configured to calculate the biological information pertaining to the test site with use of the response information acquired from the light receiver 123.

When the test site is an artery, the controller 160 is configured to calculate a pulse wave with use of the response information acquired form the sensor unit 120. A pulse wave refers to changes in volume of a blood vessel caused by inflow of blood as captured as a waveform from a body surface. That is to say, in the present embodiment, the sensor unit 120 is configured to detect the pulse wave due to changes in light intensity received by the light receiver 123. The controller 160 is configured to acquire the pulse wave as the biological information optically with use of the sensor unit 120.

In the above configuration, each sensor unit 120 is described to include the two light emitters 121 and the single light receiver 123. However, measurement may also be performed when each sensor 120 is configured to include a single light emitter 121 and the single light receiver 123. In comparison of the above configurations, precision of the measurement may be improved in the configuration with the two light emitters 121 and the single light receiver 123.

Each of the light emitters 121 emits, for example, any of green light (with a wavelength of 495 to 570 nm), red light (with a wavelength of 620 to 750 nm), and near infrared light (with a wavelength of 750 to 1600 nm). Light with a long wavelength penetrates to a deeper level of the body than light with a short wavelength. In measurement of the biological information with use of a light emitting element that emits near infrared light, measurement accuracy may be improved.

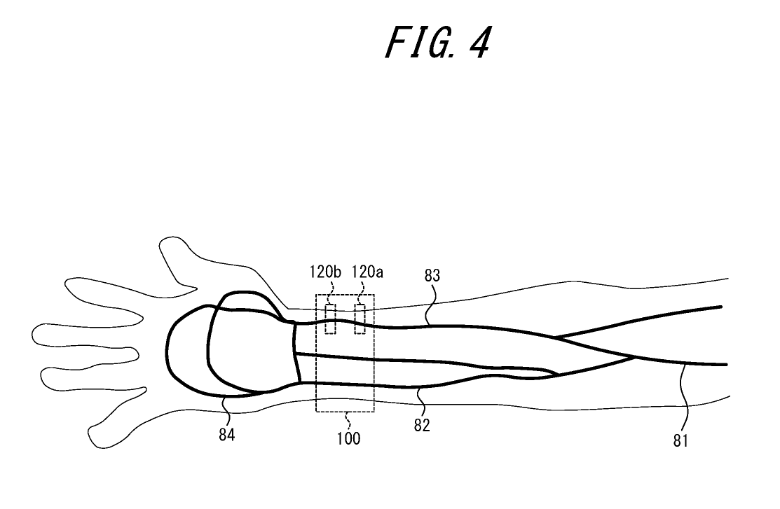

FIG. 4 is a schematic view illustrating an artery of a human right arm. In FIG. 4, a right hand palm faces to the front side. The artery of the right arm includes parts from a brachial artery 81 to a palmar artery 84 through an ulnar artery 82 and a radial artery 83. As represented by a broken line in FIG. 4, the blood pressure estimation apparatus 100 is mounted on a subject's wrist. The mounting position of the blood pressure estimation apparatus 100 is adjusted so that the first sensor unit 120a and the second sensor unit 120b are aligned with the ulnar artery 82 or the radial artery 83.

In FIG. 4, the first sensor unit 120a and the second sensor unit 120b are both disposed in alignment with the radial artery 83. The first sensor unit 120a and the second sensor unit 120b are configured to acquire pulse waves of the radial artery 83 at positions located a predetermined distance away from each other. The predetermined distance equals a distance between the first sensor unit 120a and the second sensor unit 120b.

The first sensor unit 120a and the second sensor unit 120b may both be disposed in alignment with the ulnar artery 82. In this case, the first sensor unit 120a and the second sensor unit 120b are configured to acquire pulse waves of the ulnar artery 82 at positions located the predetermined distance away from each other.

<Measurement of Pulse Wave Velocity>

In the present embodiment, a pulse wave transit velocity (which may also be called Pulse Wave Velocity: PWV) is measured by acquiring, for one artery, pulse waves at positions located the predetermined distance away from each other. For example, when the first sensor unit 120a and the second sensor unit 120b are disposed in alignment with the radial artery 83, the controller 160 calculates, for the radial artery 83, the pulse wave velocity. That is to say, the controller 160 measures the Pulse Wave Velocity (PWV) in a short wrist's distance with use of the pulse waves calculated based on the response information acquired from the first sensor unit 120a and the second sensor unit 120b.

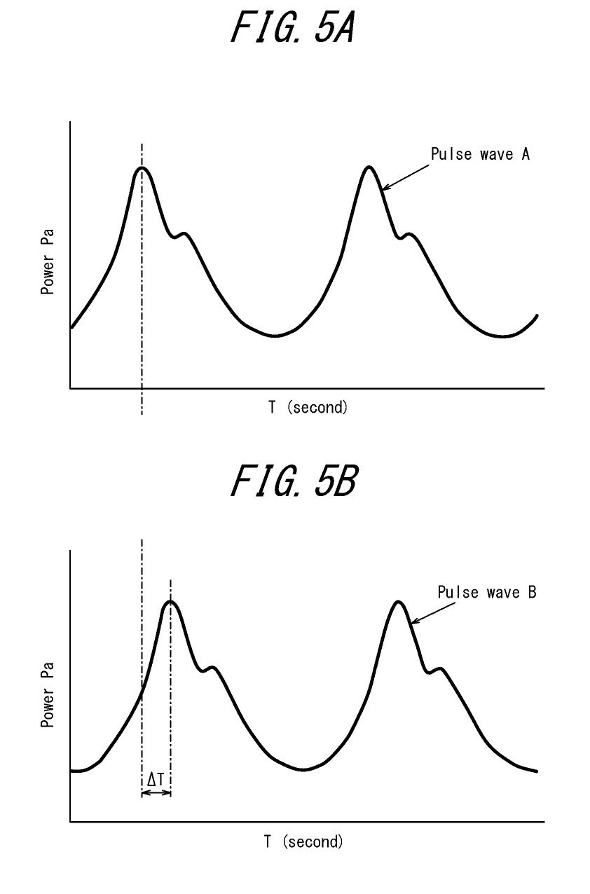

FIGS. 5A and 5B each illustrate an example of the pulse wave calculated based on the response information acquired from the corresponding sensor unit 120. The first sensor unit 120a and the second sensor unit 120b in the blood pressure estimation apparatus 100 are assumed to be disposed in alignment with the radial artery 83.

FIG. 5A illustrates a pulse wave A acquired in the first sensor unit 120a, which contacts a first test site on the radial artery 83. FIG. 5B illustrates a pulse wave B acquired in the second sensor unit 120b, which contacts a second test site on the radial artery 83. A horizontal axis and a vertical axis in each of FIGS. 5A and 5B respectively represent time and the magnitude (power) of the pulse wave. FIGS. 5A and 5B are time-synchronized with each other. In FIG. 5A, time at which the pulse wave A reaches a peak value is represented by a dashed line. In FIG. 5B, time at which the pulse wave B reaches a peak value is represented by a dashed line. In FIG. 5B, the time at which the pulse wave A reaches the peak value is also represented by a dashed line. A difference between the time corresponding to the two dashed lines in FIG. 5B equals time over which the pulse wave transits from the first test site to the second test site. The time over which the pulse wave transits form the first test site to the second test site may be also called Pulse Transit Time (PTT). In the example of FIGS. 5A and 5B, the Pulse Transit Time (PTT) is ΔT (seconds).

As illustrated in FIG. 2, the first sensor unit 120a and the second sensor unit 120b are disposed at a distance of ΔD (m). That is to say, the distance from the first test site to the second test site is ΔD (m). In this case, the pulse wave velocity (m/second) in the radial artery 83 is calculated by the following formula (1).

(PWV)=ΔD/ΔT (1)

Although the pulse wave velocity in the radial artery 83 has been described, the pulse wave velocity in the ulnar artery 82 is also calculated similarly. Furthermore, the controller 160 may calculate the pulse transit time, with time at which the pulse wave reaches a bottom value as reference. The controller 160 may also calculate the pulse transit time, with time of an inflection point at which the pulse wave rises toward the peak value as reference.

<Blood Pressure Estimation>

A pulse wave refers to a waveform representing changes in volume of a blood vessel caused by inflow of blood. The pulse wave velocity is used as an index of blood vessel stiffness. Blood vessel stiffness correlates with elevated blood pressure. That is to say, the pulse wave velocity correlates with the blood pressure. Taking advantage of the correlated relation between the pulse wave velocity and the blood pressure, the controller 160 is configured to calculate an estimated value of the subject's blood pressure from the pulse wave velocity. Generally, the relation between Blood Pressure (BP) and the Pulse Wave Velocity (PWV) is represented by the following formula (2).

(BP)=(PWV)×a+b (2)

(where a and b: coefficients)

The controller 160 may be configured to determine the coefficients (a and b) in the formula (2) by using measured values of the subject's blood pressure and the pulse wave velocities of the subject. A measured value of blood pressure may be systolic blood pressure (maximum blood pressure) or diastolic blood pressure (minimum blood pressure). A measured value of blood pressure may also be average blood pressure. The average blood pressure is represented by the following formula (3) with use of the maximum blood pressure and the minimum blood pressure.

(Average blood pressure)=(Maximum blood pressure+Minimum blood pressure×2)/3 (3)

In the present embodiment, a measured value of subject's blood pressure is measured by using a sphygmomanometer 200 (refer to FIG. 6), which is separate from the blood pressure estimation apparatus 100. FIG. 6 illustrates an example of a schematic configuration of a blood pressure estimation system 10 according to the present embodiment. The blood pressure estimation system 10 includes the blood pressure estimation apparatus 100 and the sphygmomanometer 200. The sphygmomanometer 200 may, for example, be a so-called cuff sphygmomanometer, which measures blood pressure by using a cuff (arm band) to apply pressure to an artery of a human upper arm, wrist, or the like. The sphygmomanometer 200 may be any other type of sphygmomanometer.

The blood pressure estimation apparatus 100 and the sphygmomanometer 200 may communicate with each other directly through wired or wireless communication. The blood pressure estimation apparatus 100 and the sphygmomanometer 200 may also communicate with each other via an external apparatus, such as the server 300 illustrated in FIG. 6.

<<Blood Pressure Estimation Flow>>

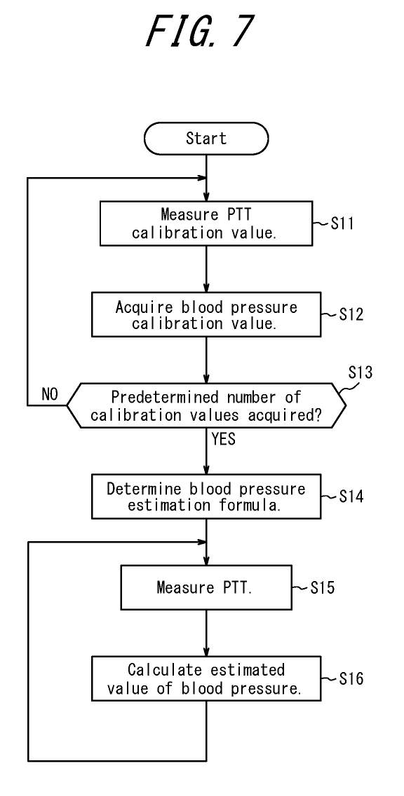

FIG. 7 is a flowchart illustrating an example of a procedure (which may also be called blood pressure estimation method) of estimating the subject's blood pressure.

Firstly, the controller 160 acquires, from the first sensor unit 120a and the second sensor unit 120b, the biological information in the first test site and the second site of the subject. Based on the biological information in the first test site and the second test site, the controller 160 calculates pulse waves as illustrated in FIGS. 5A and 5B. The controller 160 calculates the Pulse Transit Time (PTT) from the first test site to the second test site (Step S11). The controller 160 stores, in the memory 180, the Pulse Transit Time (PTT) as a PTT calibration value.

Subsequently, the controller 160 acquires, from the sphygmomanometer 200, a measured value of the subject's blood pressure (Step S12). The controller 160 stores, in the memory 180, the measured value of the blood pressure as a blood pressure calibration value.

Subsequently, the controller 160 determines whether a predetermined number of pairs of the PTT calibration value and the blood pressure calibration value have been acquired (Step S13). The predetermined number may be determined suitably. The greater the predetermined number is, the more precise the coefficients of the formula for estimating the blood pressure are. This improves precision in the blood pressure estimation.

When determining that the predetermined number of pairs of the PTT calibration value and the blood pressure calibration value have not been acquired (Step S13: NO), the controller 160 returns to Step S11.

When determining that the predetermined number of pairs of the PTT calibration value and the blood pressure calibration value have been acquired (Step S13: YES), the controller 160 determines a blood pressure estimation formula based on the PTT calibration values and the blood pressure calibration values (Step S14). The blood pressure estimation formula is determined by calculating the coefficients (a and b) in the aforementioned formula (2). When calculating the coefficients in the formula (2), the controller 160 converts the PTT calibration value to the Pulse Wave Velocity (PWV). The controller 160 may also determine a blood pressure estimation formula based on a relation between the blood pressure and the pulse transit time as represented by the following formula (4).

(BP)=(PTT)×c+d (4)

(c and d: coefficients)
In this case, the controller 160 calculates the coefficients (c and d) based on the PTT calibration values and the blood pressure calibration values.

Subsequently, the controller 160 acquires, from the first sensor unit 120a and the second sensor unit 120b, the biological information in the first test site and the second test site of the subject. Based on the biological information in the first test site and the second test site, the controller 160 calculates pulse waves as illustrated in FIGS. 5A and 5B. The controller 160 calculates the Pulse Transit Time (PTT) from the first test site to the second site (Step S15). The controller 160 stores, in the memory 180, the Pulse Transit Time (PTT).

The controller 160 calculates an estimated value of the blood pressure from the Pulse Transit Time (PTT) with use of the formula (4) (Step S16). The controller 160 may also calculate the estimated value of the blood pressure with use of the formula (2). In this case, the controller 160 converts the Pulse Transit Time (PTT) to the Pulse Wave Velocity (PWV). After calculating the estimated value of the blood pressure, the controller 160 returns to Step S15. The controller 160 repeats Steps S15 and S16 and calculates estimated values of the blood pressure continuously. The controller 160 may also end the flowchart of FIG. 7 after calculating the estimated value of the blood pressure.

As described with reference to the flowchart of FIG. 7 on the above, the controller 160 is configured to calculate the estimated value of the subject's blood pressure. With the blood pressure estimation method according to the present embodiment, unlike the blood pressure measurement using the so-called cuff sphygmomanometer, pressure is not applied to the artery. In this way, the blood pressure is estimated without causing congestion during blood pressure measurement. In the event of congestion during blood pressure measurement, blood pressure is hardly measured successively or continuously due to significant stress to the subject's body. On the other hand, the blood pressure estimation method according to the present embodiment reduces stress to the subject's body and allows continuous blood pressure estimation.

With the blood pressure estimation method according to the present embodiment, by using the pulse transit time obtained with every heartbeat, an interval between each calculation of the estimated value of the blood pressure may be one heartbeat in the shortest. In contrast, other types of measurement as in the so-called cuff sphygmomanometer that measure blood pressure by applying pressure to the artery involves the need for considering stress to the subject's body due to occurrence of congestion or the like. Accordingly, the interval between each blood pressure measurement using the cuff sphygmomanometer or the like is normally 5 minutes or more. The blood pressure estimation method according to the present embodiment reduces stress to the subject's body and accordingly, allows continuous blood pressure estimation at a regular cycle of less than 5 minutes.

[Blood Pressure Monitoring Using Blood Pressure Estimation Apparatus]

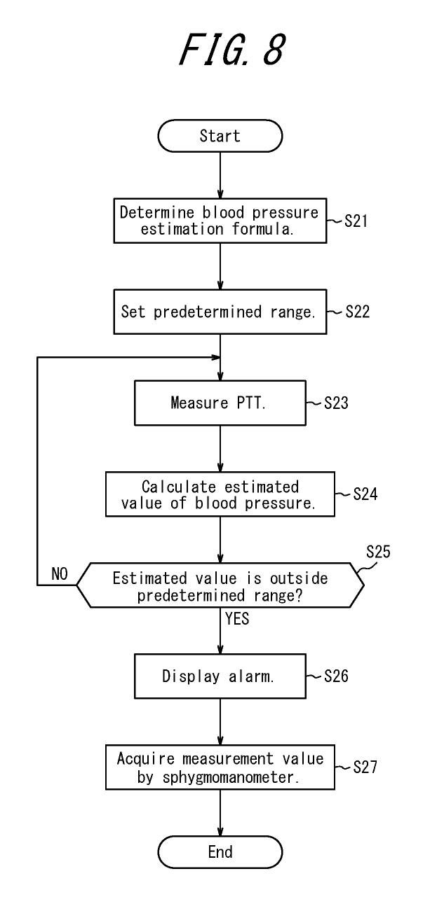

Since the blood pressure estimation apparatus 100 is configured to estimate the subject's blood pressure continuously, it may be used to monitor the subject's blood pressure. FIG. 8 is a flowchart illustrating an example of a procedure of the controller 160 to monitor an estimated value of the subject's blood pressure.

Firstly, the controller 160 determines a blood pressure estimation formula (Step S21). The controller 160 is configured to determine the blood pressure estimation formula similarly to Steps S11 through S13 in FIG. 7. The blood pressure estimation formula may be the one represented by the formula (2), which uses the Pulse Wave Velocity (PWV), or the one represented by the formula (4), which uses the Pulse Transit Time (PTT).

Subsequently, the controller 160 sets a predetermined range corresponding to the estimated value of the blood pressure (Step S22). The predetermined range may be set to a range in which the maximum blood pressure is a predetermined value or less. When the estimated value of the blood pressure is outside the predetermined range, the controller 160 notifies an alarm to the subject or the surroundings by using the display 130 or the like. The predetermined range is not limited to the range pertaining to the maximum blood pressure and may be a range pertaining to the minimum blood pressure or the average blood pressure. As an instance, a range in which the minimum blood pressure is a predetermined value or more may be set as the predetermined range. As another instance, the predetermined range may be set so that the average blood pressure is within the predetermined range.

Subsequently, similarly to Step S15 in FIG. 7, the controller 160 calculates the Pulse Transit Time (PTT) from the first test site to the second test site (Step S23).

Subsequently, the controller 160 calculates an estimated value of the blood pressure from the Pulse Transit Time (PTT) with use of the formula (2) or (4) (Step S24). The controller 160 may store, in the memory 180, the estimated value of the blood pressure.

Subsequently, the controller 160 determines whether the estimated value of the blood pressure is outside the predetermined range (Step S25). When determining that the estimated value of the blood pressure is not outside the predetermined range (Step S25: NO), the controller 160 returns to Step S23.

When determining that the estimated value of the blood pressure is outside the predetermined range (Step S25: YES), the controller 160 displays an alarm to the subject or the surroundings by using the display 130 or the like to notify that the estimated value of the pressure is outside the alarm range (Step S26). The controller 160 may also prompt the subject for measurement using the sphygmomanometer 200. Information for prompting the measurement using the sphygmomanometer 200 may be included in the alarm. In accordance with the alarm, the subject measures the blood pressure by the sphygmomanometer 200. Alternatively, in accordance with the alarm, the controller 160 may measure the subject's blood pressure automatically.

Subsequently, the controller 160 acquires a measured value of the subject's blood pressure measured by the sphygmomanometer 200 (Step S27). The controller 160 may store, in the memory 180, the measured value that is acquired.

After acquiring the measured value of the subject's blood pressure, the controller 160 ends the flowchart of FIG. 8. After acquiring the measured value of the subject's blood pressure, the controller 160 may also return to Step S21. In this case, the controller 160 may calculate the coefficients of the blood pressure estimation formula again by using the measured value measured by the sphygmomanometer 200. After acquiring the measured value of the subject's blood pressure, the controller 160 may also return to Step S23. In this case, the controller 160 estimates the subject's blood pressure continuously.

As described with reference to the flowchart of FIG. 8 on the above, the controller 160 is configured to monitor the estimated value of the subject's blood pressure.

[Mounting of Blood Pressure Estimation Apparatus]

The subject conducts measurement by using the blood pressure estimation apparatus 100, which is mounted, for example, on the wrist. When the biological information that the blood pressure estimation apparatus 100 acquires corresponds to a pulse wave, the test site is the ulnar artery 82 or the radial artery 83. When the blood pressure estimation apparatus 100 is mounted on the subject, the position of each sensor unit 120 is adjusted so that the measuring light emitted from the light emitters 121 of the sensor unit 120 irradiates the ulnar artery 82 or the radial artery 83.

The blood pressure estimation apparatus 100 is mounted on the subject, with the sensor unit 120 contacting the test site, such as the wrist. Especially, it may be for the subject himself/herself to adjust the sensor unit 120 during mounting to a position in contact with the wrist by which the ulnar artery 82 or the radial artery 83 is irradiated with the measuring light.

With the blood pressure estimation apparatus 100 being mounted, each sensor unit 120 is mounted on the subject while being tightly contacted to the wrist by elastic force of the sensor holder 110 or the mounted portion 140. By the sensor unit 120 being tightly contacted to the wrist, a positional relation between the wrist and the sensor unit 120 is unlikely to change, and this improve precision in measurement in the sensor unit 120.

The sensor units 120 may also be supported in a manner such that the sensor units 120 are displaceable with respect to the sensor holder 110 independently. By dosing so, the sensor units 120 are more likely to be tightly contacted to the wrist, which is the test site. Furthermore, when the sensor holder 110 is offset from the wrist, the sensor units 120, which each are displaceable, tend to maintain the state in which the sensor units 120 are closely contacted to the wrist. Accordingly, the positional relation between each sensor unit 120 and the wrist is unlikely to change, and conditions of measurement of the biological information by the sensor unit 120 are unlikely to change. Thus, precision in measurement of the biological information is improved.

Moreover, each sensor unit 120 is configured not to contact the wrist with pressure that is greater than or equal to predetermined pressure, in the state where the blood pressure estimation apparatus 100 is mounted. The predetermined pressure is determined suitably based on the biological information measured by the blood pressure estimation apparatus 100, the configuration of the blood pressure estimation apparatus 100, and the like. The predetermined pressure may be pressure with which an error is unlikely to occur in a result of measurement of the biological information. In the present embodiment, the blood pressure estimation apparatus 100 measures the pulse wave velocity as the biological information. Accordingly, the predetermined pressure may be pressure with which an error is unlikely to occur in a result of measurement of the pulse wave velocity.

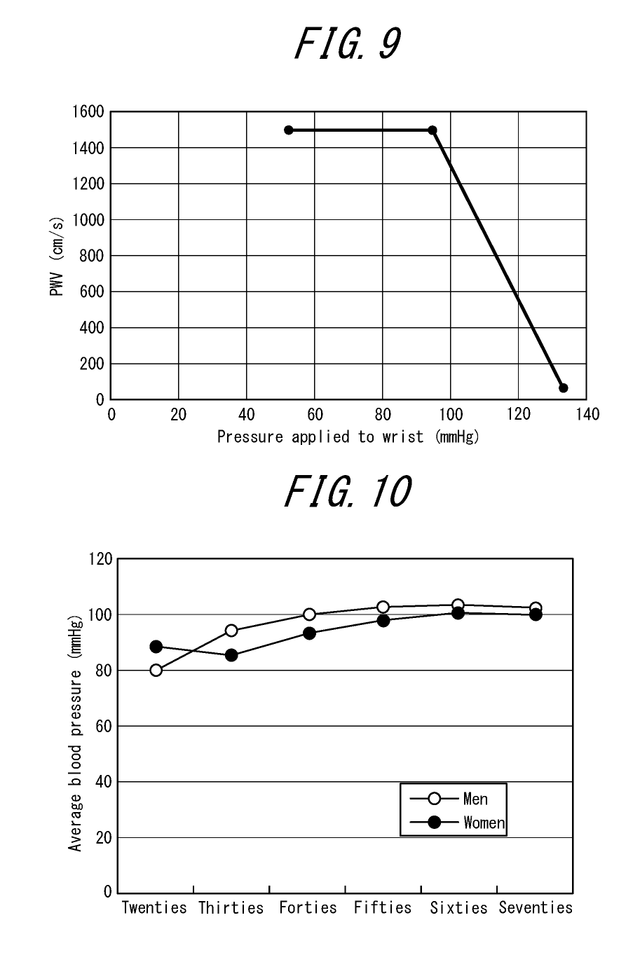

With reference to FIG. 9, a description is given of a value that may be as the predetermined pressure in the blood pressure estimation apparatus 100 according to the present embodiment. FIG. 9 is a graph illustrating a result of experiment regarding a relation between contact pressure applied by each sensor unit 120 to the wrist and the PWV when the blood pressure estimation apparatus 100 is mounted on the wrist. The graph of FIG. 9 illustrates the result of experiment conducted on the subject whose average blood pressure is approximately 95 mmHg.

As illustrated in FIG. 9, when the contact pressure applied from the sensor unit 120 to the wrist equals the subject's average blood pressure (approximately 95 mmHg), a difference in pressure between blood pressure inside a blood vessel and the contact pressure applied to the blood vessel externally is on average zero. Accordingly, blood flow is unlikely to be affected by the contact pressure. Consequently, the result of measurement of the pulse wave velocity by the blood pressure estimation apparatus 100 tends to be improved.

When the contact pressure is less than the subject's average blood pressure (which is approximately 95 mmHg in FIG. 9), expansion and contraction of a blood vessel wall is unlikely to be affected by a change in the contact pressure. In this case, elasticity of the blood vessel wall remains substantially constant, and a substantially constant result of measurement of the pulse wave velocity is obtained. The contact pressure may be less than the subject's average blood pressure. For example, the sensor unit 120 does not need to be in contact with the wrist.

When the contact pressure is greater than the subject's average blood pressure (which is approximately 95 mmHg in FIG. 9), expansion and contraction properties of the blood vessel wall is likely to be affected by the contact pressure. Consequently, the pulse wave velocity is decreased as the contact pressure is increased.

According to the result of experiment illustrated in FIG. 9, measurement accuracy of the pulse wave velocity tends not to be degraded when the contact pressure is less than or equal to the subject's average blood pressure (which is approximately 95 mmHg in FIG. 9). On the other hand, the measurement accuracy tends to be degraded when the contact pressure is greater than the subject's average blood pressure. Accordingly, the sensor unit 120 may contacts the test site with pressure that is less than or equal to the subject's average blood pressure as the predetermined pressure, or, may be out of contact with the test site.

FIG. 10 illustrates average blood pressure separated by age, based on a result of the Fifth Basic Survey of Cardiovascular Diseases published by the Ministry of Health. To make the blood pressure estimation apparatus 100 versatile, the blood pressure estimation apparatus 100 may be usable by adults of at least twenty years old. The measurement accuracy tends not to be degraded when the contact pressure is less than or equal to the subject's average blood pressure. Approximately 80 mmHg, which is the average blood pressure of men in their twenties who have the lowest blood pressure in FIG. 10, may be set as the predetermined pressure in the blood pressure estimation apparatus 100 according to the present embodiment. Accordingly, the blood pressure estimation apparatus 100 may be configured to allow, when being mounted, each sensor unit 120 to contact the corresponding test site with pressure that is less than or equal to 80 mmHg. In this case, the measurement accuracy of the pulse wave velocity is improved.

In the present embodiment, the first sensor unit 120a and the second sensor unit 120b contact the test sites located at a predetermined distance from each other in a direction of a predetermined blood vessel of the subject. The first sensor unit 120a and the second sensor unit 120b may contact the test sites located at the predetermined distance (ΔD), with the Y axis direction in FIG. 2 extending along the direction of the predetermined blood vessel of the subject.

In the present embodiment, in one of the test sites, in contact with the first sensor unit 120a and the second sensor unit 120b, that is located closer to a subject's heart along the predetermined blood vessel, the contact pressure of the corresponding sensor unit 120 may be less than the predetermined pressure. Suppose, for example, that, when the blood pressure estimation apparatus 100 is mounted on the subject's wrist, the positive Y axis direction in FIG. 2 is directed toward the subject's upper arm, and the negative Y axis direction in FIG. 2 is directed toward the subject's palm. In this case, a distance from one of the test sites that is located further in the positive Y axis direction (further toward the subject's upper arm) to the subject's heart along the predetermined blood vessel is less than a distance from the other test site that is located further in the negative Y axis direction (further toward the subject's palm) to the subject's heart along the predetermined blood vessel. In this case, therefore, the first sensor unit 120a, which contacts the corresponding test site further in the positive Y axis direction (further toward the subject's upper arm), may contact the test site with pressure that is less than the predetermined pressure.

The blood pressure estimation system 10 and the blood pressure estimation apparatus 100 according to the present embodiment measure the pulse wave velocity of the subject by using the sensor units 120, which are each mounted with pressure that is less than the predetermined pressure. That is to say, in the present embodiment, the pulse wave velocity of the subject is measured without applying the predetermined pressure. The predetermined pressure may be pressure that is applied during blood pressure measurement using the so-called cuff sphygmomanometer. Since the pulse wave velocity is measured with pressure that is less than the pressure applied by the cuff sphygmomanometer, the subject's blood pressure is estimated without applying pressure to the artery like the cuff sphygmomanometer. The blood pressure estimation system 10 and the blood pressure estimation apparatus 100 according to the present embodiment allow continuous calculation of the estimated value of the subject's blood pressure without applying the predetermined pressure to the subject.

The blood pressure estimation system 10 and the blood pressure estimation apparatus 100 according to the present embodiment measure the pulse wave velocity of the subject by using the plurality of sensor units 120. This prevents, even when medicine such as a hypotensive drug that changes cardiac contractility is used, the measured value of the pulse wave velocity to be affected by the medication.

The blood pressure estimation system 10 and the blood pressure estimation apparatus 100 according to the present embodiment measure the pulse wave velocity of the subject by using the plurality of sensor units 120, which are disposed at a short distance on, for example, the ulnar artery 82 or the radial artery 83 of the wrist. This makes the configuration of each sensor unit 120 compact. The blood pressure estimation apparatus 100 according to the present embodiment has merits, such as excellent portability or daily mountability.

The blood pressure estimation system 10 and the blood pressure estimation apparatus 100 according to the present embodiment estimate the subject's blood pressure by using pulse waves detected from a thick blood vessel, such as the ulnar artery 82 or the radial artery 83 of the wrist. The blood pressure estimation system 10 and the blood pressure estimation apparatus 100 according to the present embodiment allow blood pressure estimation of the subject even when the subject is in peripheral circulatory failure.

As illustrated in FIG. 6, the blood pressure estimation apparatus 100 may be connected to an external apparatus, such as the server 300. The controller 160 may output, to the server 300, various data, such as the pulse transit time, the estimated value of the blood pressure, or the measured value of the blood pressure. The sphygmomanometer 200 may output, to the server 300, various data, such as the measured value of the blood pressure. In this case, the server 300 stores the various data acquired from the controller 160 or the sphygmomanometer 200. The controller 160 acquires, from the server 300, the various data stored in the server 300. The controller 160 may also acquire the measured value of the blood pressure measured by the sphygmomanometer 200 via the server 300.

### Embodiment 2

The blood pressure estimation system 10 described in Embodiment 1 includes the sphygmomanometer 200, which is a separate body. In Embodiment 2, a description is given of a configuration in which the blood pressure estimation apparatus 100 and the sphygmomanometer 200 are integrated.

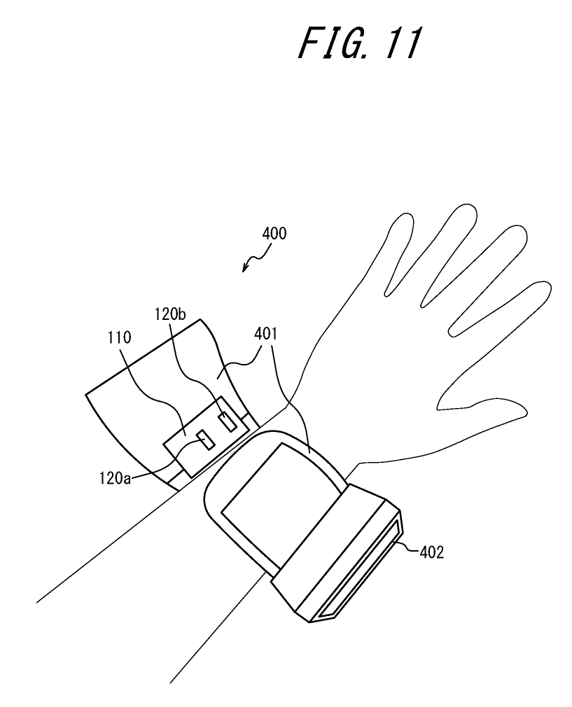

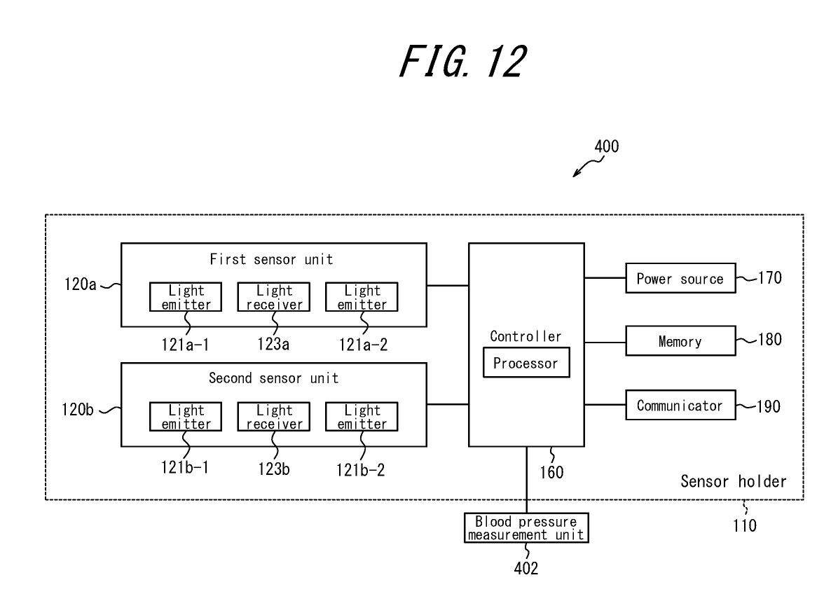

FIG. 11 is a schematic view of a sphygmomanometer 400, which is equipped with pulse wave sensors, according to Embodiment 2. The sphygmomanometer 400, which is equipped with the pulse wave sensors, may simply be called sphygmomanometer 400. The sphygmomanometer 400 includes a cuff 401 and a blood pressure measurement unit 402. In the cuff 401, the sensor holder 110 of the blood pressure estimation apparatus 100 according to Embodiment 1 is disposed. The sensor holder 110 includes the first sensor unit 120a and the second sensor unit 120b. In the present embodiment, the sensor units 120 each serve as a pulse wave sensor that detects a pulse wave.

FIG. 12 is a functional block diagram illustrating an example of a schematic configuration of the sphygmomanometer 400 illustrated in FIG. 11. Similarly to FIG. 3, the sphygmomanometer 400 includes the first sensor unit 120a, the second sensor unit 120b, the controller 160, the power source 170, the memory 180, and the communicator 190. The sphygmomanometer 400 further includes the blood pressure measurement unit 402. The blood pressure measurement unit 402 is connected to the controller 160.

As illustrated in FIG. 12, the controller 160, the power source 170, the memory 180, and the communicator 190 are included in the sensor holder 110. The blood pressure measurement unit 402 may be included in the sensor holder 110. An entirety or a part of the controller 160, the power source 170, the memory 180, and the communicator 190 may also be included in the blood pressure measurement unit 402 instead of the sensor holder 110. The first sensor unit 120a, the second sensor unit 120b, the controller 160, the power source 170, the memory 180, and the communicator 190 have the same configurations as those described in Embodiment 1.

The sphygmomanometer 400 is mounted on a test site, such as the wrist, of the subject. The cuff 401 is wound around the test site. When the cuff 401 is wound around the test site, the position of the sensor holder 110 is adjusted so that the first sensor unit 120a and the second sensor unit 120b, which are disposed in the cuff 401, are aligned with the artery in the test site. The cuff 401 is provided with a marking or the like in correspondence with the position in which the sensor holder 110 is to be disposed. The position of the sensor holder 110 is easily adjusted with reference to the marking. The blood pressure measurement unit 402 measures blood pressure by applying pressure to the artery in the test site with use of the cuff 401.

The controller 160 outputs, to the blood pressure measurement unit 402, control information instructing blood pressure measurement. In response to the response information received from the controller 160, the blood pressure measurement unit 402 measures the subject's blood pressure. The blood pressure measurement unit 402 may also acquire, from the subject or the like, input instructing blood pressure measurement. In this case, regardless of the control information from the controller 160, the blood pressure measurement unit 402 measures the blood pressure in response to the input instructing blood pressure measurement. The blood pressure measurement unit 402 outputs, to the controller 160, a measured value of the subject's blood pressure. The controller 160 stores, in the memory 180, the measured value of the subject's blood pressure.

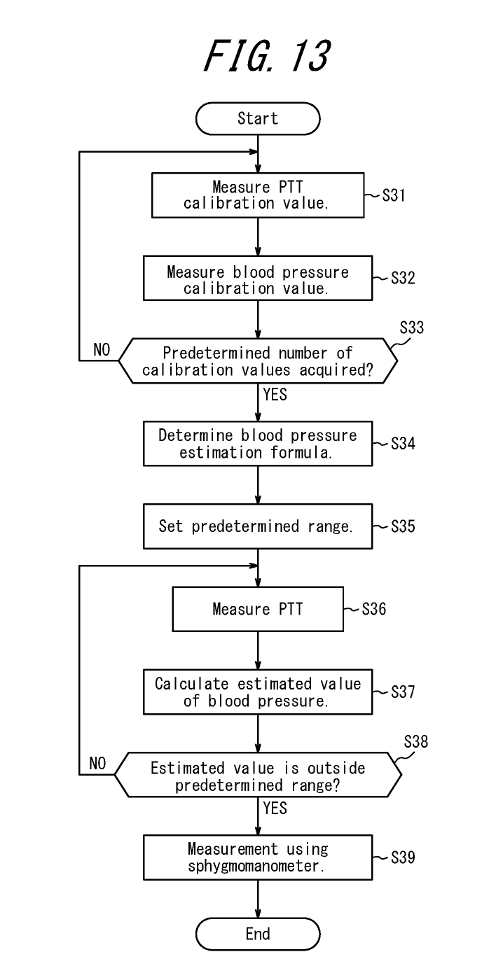

Similarly to the blood pressure estimation apparatus 100 according to Embodiment 1, the controller 160 is configured to calculate an estimated value of the subject's blood pressure. The controller 160 is also configured to monitor the subject's blood pressure. The sphygmomanometer 400 according to Embodiment 2 differs from the blood pressure estimation apparatus 100 according to Embodiment 1 in that the sphygmomanometer 400 includes the blood pressure measurement unit 402. FIG. 13 is a flowchart illustrating an example of a procedure of monitoring the subject's blood pressure by the sphygmomanometer 400 illustrated in FIG. 11. Regarding steps that are substantially the same as those in the flowcharts of FIGS. 7 and 8, a description is omitted as needed.

Firstly, the controller 160 acquires, from the first sensor unit 120a and the second sensor unit 120b, the biological information in the first test site and the second site. Based on the biological information in the first test site and the second test site, the controller 160 calculates pulse waves as illustrated in FIGS. 5A and 5B. The controller 160 calculates the Pulse Transit Time (PTT) from the first test site to the second site (Step S31). The controller 160 stores, in the memory 180, the Pulse Transit Time (PTT) as a PTT calibration value. Step S31 is substantially the same as Step S11 in FIG. 7.

Subsequently, the controller 160 measures the subject's blood pressure by using the blood pressure measurement unit 402 (Step S32). The controller 160 stores, in the memory 180, the measured value of the blood pressure as a blood pressure calibration value.

Subsequently, the controller 160 determines whether a predetermined number of pairs of the PTT calibration value and the blood pressure calibration value have been acquired (Step S33). Step S33 is substantially the same as Step S13 in FIG. 7. Regarding the predetermined number, refer to the description with respect to Step S13 in FIG. 7. When determining that the predetermined number of pairs of the PTT calibration value and the blood pressure calibration value have not been acquired (Step S33: NO), the controller 160 returns to Step S31.

When determining that the predetermined number of pairs of the PTT calibration value and the blood pressure calibration value have been acquired (Step S33: YES), the controller 160 determines a blood pressure estimation formula based on the PTT calibration values and the blood pressure calibration values (Step S34). Step S34 is substantially the same as Step S14 in FIG. 7.

Subsequently, the controller 160 determines a predetermined range corresponding to the estimated value of the blood pressure (Step S35). Step S35 is substantially the same as Step S22 in FIG. 8. Regarding the predetermined range, refer to the description with respect to Step S22 in FIG. 8.

Subsequently, similarly to Step S15 in FIG. 7, the controller 160 calculates the Pulse Transit Time (PTT) from the first test site to the second test site (Step S36).

Subsequently, the controller 160 calculates an estimated value of the blood pressure from the Pulse Transit Time (PTT) with use of the formula (2) or (4) (Step S37). The controller 160 may store, in the memory 180, the estimated value of the blood pressure.

Subsequently, the controller 160 determines whether the estimated value of the blood pressure is outside the predetermined range (Step S38). When determining that the estimated value of the blood pressure is not outside the predetermined range (Step S38: NO), the controller 160 returns to Step S36. In this case, the controller 160 estimates the subject's blood pressure continuously.

When determining that the estimated value of the blood pressure is outside the predetermined range (Step S38: YES), the controller 160 measures the subject's blood pressure by using the blood pressure measurement unit 402 (Step S39). The controller 160 may store, in the memory 180, the measured value of the blood pressure.

After acquiring the measured value of the subject's blood pressure, the controller 160 ends the flowchart of FIG. 13. After acquiring the measured value of the subject's blood pressure, the controller 160 may also return to Step S31. In this case, the controller 160 may calculate the coefficients of the blood pressure estimation formula again by using the measured value of the blood pressure measured in Step S39. After acquiring the measured value of the subject's blood pressure, the controller 160 may also return to Step S36. In this case, the controller 160 estimates the subject's blood pressure continuously.

As described with reference to the flowchart of FIG. 13 on the above, the sphygmomanometer 400 according to Embodiment 2 is configured to monitor the subject's blood pressure. The sphygmomanometer 400 according to the present embodiment reduces a difference in time between time at which the pulse transit time is measured by using the sensor units 120 and time at which the blood pressure is measured by using the blood pressure measurement unit 402. Accordingly, precision in calculation of the estimated value of the subject's blood pressure based on the pulse transit time is improved.

The sphygmomanometer 400 according to the present embodiment monitors the estimated value of the blood pressure that is based on the pulse transit time. When the estimated value of the blood pressure is outside the range, the blood pressure measurement using the blood pressure measurement unit 402 is automatically conducted. This omits the need for the subject to be aware of timing of blood pressure measurement. Accordingly, the subject is freed from bothersomeness of blood pressure measurement.

The sphygmomanometer 400 according to the present embodiment includes the sensor units 120, which each detect a pulse wave, and the blood pressure measurement unit 402, which measures blood pressure. Thus configured sphygmomanometer 400 has excellent portability. Furthermore, thus configured sphygmomanometer 400 allows daily blood pressure estimation or measurement.

Note that the present disclosure is not limited to the embodiments set forth above and may be modified or varied in a multiple ways. For example, functions and the like included in various component parts and the like may be rearranged as long as the functions and the like are logically consistent. A plurality of component parts and the like may also be integrated or separated.

For example, in the above embodiments, the blood pressure estimation apparatus 100 is described to include the two sensor units 120, namely, the first sensor unit 120a and the second sensor unit 120b. However, the number of the sensor units 120 herein is not limited to two and may be any number that is greater than or equal to two. In this case, the shape of the sensor holder 110 may be changed as needed in accordance with the number of the sensor units 120.

In the above embodiments, each sensor unit 120 is described to include the light emitters 121 and the light receiver 123. The sensor unit 120 is not limited to an optical sensor. The sensor unit 120 may be a gyro sensor. The gyro sensor is disposed in alignment with the ulnar artery 82 or the radial artery 83 of the wrist. The gyro sensor is configured to detect a pulsation of the ulnar artery 82 or the radial artery 83 of the wrist as an acceleration. A pulse wave may be detected by using data pertaining to the acceleration detected by the gyro sensor. The sensor unit 120 may be a displacement sensor including a piezoelectric element or the like. The displacement sensor is configured to detect the pulsation of the ulnar artery 82 or the radial artery 83 as displacement. A pulse wave may be detected by using data pertaining to the displacement detected by the displacement sensor.

The blood pressure estimation apparatus 100 may include a notification unit that notifies the subject of a result of measurement of the biological information. The notification unit may notify the subject by any method that allows the user to perceive. The notification unit may notify the subject by, for example, sound, an image, vibration, or a combination thereof. The method of notification by the notification unit is not limited to the above examples.

In the above embodiments, the blood pressure estimation apparatus 100 is described to be wound around the subject's wrist for use. The use mode of the blood pressure estimation apparatus 100 is not limited to the above. The blood pressure estimation apparatus 100 may be wound around a body part, such as an ankle, other than the wrist depending on the positions of the test sites.

In the above embodiment, the blood pressure estimation apparatus 100 is described to estimate the blood pressure from the pulse wave velocity that is acquired. The blood pressure estimation apparatus 100, which is configured to acquire a pulse wave with high precision, may measure the biological information based on the pulse wave. The blood pressure estimation apparatus 100 may be configured to measure the blood pressure from the acquired pulse wave. The blood pressure estimation apparatus 100 may also be configured to measure a pulse from the acquired pulse wave.

1. A blood pressure estimation apparatus, comprising:
a plurality of sensors that each detects a pulse wave of a subject without applying, to the subject, pressure that is greater than predetermined pressure; and
a controller that calculates an estimated value of subject's blood pressure continuously, based on a measured value of the subject's blood pressure acquired from a sphygmomanometer and on output of the sensors.

2. The blood pressure estimation apparatus of claim 1, wherein
the measured value of subject's blood pressure is acquired from a cuff sphygmomanometer, and
the predetermined pressure is a pressure that the cuff sphygmomanometer applies during blood pressure measurement.

3. The blood pressure estimation apparatus of claim 1, wherein, when the estimated value is outside a predetermined value range, the controller notifies that the estimated value is outside the predetermined value range.

4. The blood pressure estimation apparatus of claim 1, wherein, when the estimated value is outside a predetermined value range, the controller prompts the subject to measure blood pressure.

5. A sphygmomanometer, comprising:
a plurality of sensors that each detects a pulse wave of a subject without applying, to the subject, pressure that is greater than predetermined pressure;
a blood pressure measurement unit; and
a controller that calculates an estimated value of subject's blood pressure continuously, based on a measured value of the subject's blood pressure acquired from the blood pressure measurement unit and on output of the sensors.

6. The sphygmomanometer of claim 5, wherein the blood pressure measurement unit measures the subject's blood pressure when the estimated value is outside a predetermined value range.

7. The sphygmomanometer of claim 5, wherein the plurality of sensors is disposed in the blood pressure measurement unit.

8. A blood pressure estimation system, comprising:
a sphygmomanometer; and a blood pressure estimation apparatus, wherein
the blood pressure estimation apparatus comprises:
a plurality of sensors that each detects a pulse wave of a subject without applying, to the subject, pressure that is greater than predetermined pressure; and
a controller that calculates an estimated value of subject's blood pressure continuously, based on a measured value of the subject's blood pressure acquired from the sphygmomanometer and on output of the sensors.

9. A blood pressure estimation method, comprising:
detecting a pulse wave of a subject by a plurality of sensors without applying, to the subject, pressure that is greater than predetermined pressure;
calculating an estimated value of subject's blood pressure continuously, based on a measured value of the subject's blood pressure acquired from a sphygmomanometer and on output of the sensors.
